SEQUENCE LISTING (20 sequences)

SEQ ID NO: 1 - protein, 54 aa, unidentified
  REGION 1..54: note = None
DGVKLCDVPSGTWSGHCGSSSKCSQQCKDREHFAYGGACHYQFPSVKCFCKRQC

SEQ ID NO: 2 - protein, 51 aa, unidentified
  REGION 1..51: note = None
QKLCQRPSGTWSGVCGNNNACKNQCIRLEKARHGSCNYVFPAHKCICYFPC

SEQ ID NO: 3 - protein, 50 aa, unidentified
  REGION 1..50: note = None
LCNERPSQTWSGNCGNTAHCDKQCQDWEKASHGACHKRENHWKCFCYFNC

SEQ ID NO: 4 - protein, 45 aa, unidentified
  REGION 1..45: note = None
RECKAQGRHGTCFRDANCVQVCEKQAGWSHGDCRAQFKCKCIFEC

SEQ ID NO: 5 - protein, 52 aa, unidentified
  REGION 1..52: note = None
MLCKLSMFGAVLGVPACAIDCLPMGKTGGSCEGGVCGCRKLTFKILWDKKFG

SEQ ID NO: 6 - protein, 38 aa, unidentified
  REGION 1..38: note = None
GFGCPLNQGACHNHCRSIKRRGGYCSGIIKQTCTCYRK

SEQ ID NO: 7 - protein, 41 aa, unidentified
  REGION 1..41: note = None
APGNKAECEREKGYCGFLKCSFPFVVSGKCSRFFFCCKNIW

SEQ ID NO: 8 - protein, 45 aa, unidentified
  REGION 1..45: note = None
FLPKTLRKFFCRIRGGRCAVLNCLGKEEQIGRCSNSGRKCCRKKK

SEQ ID NO: 9 - protein, 50 aa, unidentified
  REGION 1..50: note = None
RMCKTPSGKFKGYCVNNTNCKNVCRTEGFPTGSCDFHVAGRKCYCYKPCP

SEQ ID NO: 10 - protein, 41 aa, unidentified
  REGION 1..41: note = None
SPAIWGCDSFLGYCRLACFAHEASVGQKECAEGMLCCIPNV

SEQ ID NO: 11 - protein, 64 aa, unidentified
  REGION 1..64: note = None
ADIDFSTCARMDVPILKKAAQGLCITSCSMQNCGTGSCKKRSGRPTCVCYRCANGGGDIPLGAL

SEQ ID NO: 12 - protein, 53 aa, unidentified
  REGION 1..53: note = None
SCPGLCAKRSKTWSGWCGSSNNCDKQCRTKEGATHGACHGNILKRACDCYFKC

SEQ ID NO: 13 - protein, 45 aa, unidentified
  REGION 1..45: note = None
KTCENLADKYRGPCFSGCDTHCTTKENAVSGRCRDDFRCWCTKRC

SEQ ID NO: 14 - protein, 48 aa, unidentified
  REGION 1..48: note = None
RTCESQSHRFKGPCARKANCASVCNTEGFPDGYCHGVRRRCMCTKPCP

SEQ ID NO: 15 - protein, 36 aa, unidentified
  REGION 1..36: note = None
ACNFQSCWATCQAQHSIYFRRAFCDRSQCKCVFVRG

SEQ ID NO: 16 - protein, 30 aa, unidentified
  REGION 1..30: note = None
VGECVRGRCPSGMCCSQFGYCGKGPKYCGR

SEQ ID NO: 17 - protein, 45 aa, unidentified
  REGION 1..45: note = None
QQCGRQAGNRRCANNLCCSQYGYCGRTNEYCCTSQGCQSQCRRCG

SEQ ID NO: 18 - protein, 63 aa, unidentified
  REGION 1..63: note = None
GSNFCDSKCKLRCSKAGLADRCLKYCGICCEECKCVPSGTYGNKHECPCYRDKKNSKGKSKCP

SEQ ID NO: 19 - protein, 38 aa, unidentified
  REGION 1..38: note = None
AKCIKNGKGCREDQGPPFCCSGFCYRQVGWARGYCKNR

SEQ ID NO: 20 - protein, 46 aa, unidentified
  REGION 1..46: note = None
KSCCPNTTGRNIYNACRLTGAPRPTCAKLSGCKIISGSTCPSDYPK

(19) United States
(12) Patent Application Publication
(10) Pub. No.: US 2024/0245059 A1
(43) Pub. Date: Jul. 25, 2024

(54) LIPOSOMES CONTAINING SYNERGISTIC ANTIMICROBIAL COMPOSITIONS BASED ON SELECTED PEPTIDES AND FATTY ACIDS

(71) Applicant: CLEVER BIOSCIENCE S.R.L., Casanova Lonati (PV) (IT)

(72) Inventors: Emma MAZZEI, Milano (IT); Elisa BREVIARIO, Bonate Sopra (BG) (IT); Stefano ZUCCHINALI, Pontirolo Nuovo (BG) (IT); Giorgio FRESCHI, Piacenza (IT); Davide DE SIMEIS, Milazzo (ME) (IT)

(73) Assignee: CLEVER BIOSCIENCE S.R.L., Casanova Lonati (PV) (IT)

(21) Appl. No.: 18/576,928

(22) PCT Filed: Jul. 14, 2022
(86) PCT No.: PCT/EP2022/069723

§ 371 Date: Jan. 5, 2024

(30) Foreign Application Priority Data
Jul. 14, 2021 (IT) 102021000018542

(51) Int. Cl. A01N 63/50; A01N 25/04; A01P 1/00; A01P 3/00

(52) U.S. Cl. CPC A01N 63/50; A01N 25/04; A01P 1/00; A01P 3/00

(57) ABSTRACT

Novel synergistic combinations based on antimicrobial peptides and fatty acids and formulated in liposomes are described. Antimicrobial peptides may be selected from the classes of defensins, thionins, heveins, snakins/GASA, knottins. Fatty acids may contain 4 to 22 carbon atoms and may be saturated, monounsaturated or polyunsaturated. The present peptides and fatty acids synergize, thereby providing a strong antifungal and antibacterial activity, with important applications, especially in the agronomic field.

### FIELD OF THE INVENTION

The present invention pertains the field of antimicrobial products and compositions containing the same, in particular for the use in the agronomic field.

### PRIOR ART

Many plants have an advantageous, often essential, relationship with the microorganisms of the environment (water, soil, air). However, this relationship may become unbalanced: in this case, the microorganisms, in particular fungi and bacteria, transform into parasites and kill the plants by depriving them of nutritious substances. For example, fungal infections destroy every year more than 125 million tons of crops worldwide.

These contaminations are mainly dealt with antimicrobial synthetic products; however, the latter entail many problems: they kill also microorganisms that are beneficial for the soil, thereby causing serious consequences for plants. Moreover, the synthetic antimicrobial agents cause several environmental effects, in addition to toxicological problems for humans who come in contact with them. These problems are particularly relevant and urgent in the agronomic field, considering the wide surfaces to be treated and the corresponding massive use of antimicrobial agents, which is required to ensure sufficient product concentrations in the vicinity of all the plants to be treated. Further problems are those related to the appearance of resistant strains, with the consequent growing need of newer antimicrobial products with high efficacy are required.

In recent years, the regulations for environment protection have imposed limits to the use of synthetic antimicrobial agents, supporting at the same time the research of new products with natural characteristics, which should be less toxic for humans and easier to dispose of. The use of antimicrobial peptides, i.e. small protein molecules consisting of 10-100 amino acids, broadly occurring in nature (in bacteria, plants, insects, etc.) is very interesting. Currently, about 800 substances classified as antimicrobial peptides are known. The first to be studied were cecropins, isolated from silkworm (Hyalophora cecropia) in the early 1980s, and melittin, isolated from the venom of honeybee (Apis mellifera). The latter is one of the peptides studied most thoroughly and is therefore often used as a reference for studying new molecules. The skin of several amphibian species is a rich source of peptides (bombesins, magainins, temporins, etc.), produced and secreted by granular glands in response to a variety of stimuli. In humans and in other mammals (mouse, rat, rabbit), antimicrobial peptides belonging to the defensin family are stored in the form of granules in neutrophils (blood cells specialized in phagocytosis), whereas polymorphonuclear leukocytes of bovines are rich in peptides belonging to the cathelicidin family that showed in vitro and in vivo a significant antimicrobial activity.

Antimicrobial peptides have an action spectrum that is quite aspecific and thus generally broad against viruses, bacteria, fungi and protozoans; the activity arises rapidly and extends to microorganisms that have developed resistance. The action mechanism is attributable to the alteration of cellular membranes, with effects such as disorganization of membrane structure, alteration of permeability, outflow of cytoplasm components and cell lysis (destruction). Some peptides, such as buforin, directly interact with intracellular targets (DNA and/or RNA) inhibiting functions that are vital for the cell. Other peptides (for example those derived from cathelicidins and defensins) inhibit pro-inflammatory and immune-defense response of the host organism.

The low selectivity of these compounds on one hand widens the action spectrum; on the other hand, it involves a non-tailored interaction with the concerned microorganism, resulting in significant variations of potency across different microorganisms, making it difficult to maintain an average high level of activity against a large group of target microorganisms. A particularly resistant sub-group of microorganisms is that of phytoplasma, i.e. special bacteria without cell wall: they access the inner parts of the plant (phloema) via vector insects and cause serious diseases, even lethal for the plant; to date there are no curative strategies to contrast phytoplasma: in fact, the traditional antibacterial strategies, aimed to hit the bacterial cell wall, are ineffective on these microorganisms and the available treatments are limited to preventive ones.

The low specificity of antimicrobial peptides also increases the risk of undesired effects on the plant and/or on humans. The possibility of reducing the amount/concentration of these products is not effectively viable since this is associated to an undesired reduction of treatment efficacy. Therefore, there is still the need for new antimicrobial products and compositions that associate the advantage of a broad action spectrum to the advantage of a stronger activity, such as to allow the use in amounts lower than the standard ones without compromising the extent of the effect. In particular, there is still the need for synergistic compositions that allow to obtain a higher antimicrobial effect than the sum of those obtainable by the components constituting the composition, taken separately.

Another problem faced by the antimicrobial treatments in the agronomic field, particularly when the products are applied by irroration, lies in that the efficacy of the applied product is undesirably reduced due leaching from the plant surfaces caused by rainwater and/or humidity. No satisfactory solutions to this problem have been so far proposed. In particular, the use of bioadhesive ingredients is not advisable, since they may remain on the leaves surfaces hindering the vital gas exchange processes taking place there; increasing the lipophilic character of the product is also not a satisfactory solution: in fact, insofar as lipophilic properties are opposed to water, they may also prevent a proper adhesion to the naturally wet plant surfaces during application. Liposomes have been so far proposed in the pharmaceutical and cosmetic field to increase the water solubility of highly lipophilic products.

### SUMMARY

It has been now discovered that it is possible to obtain an unexpectedly high antimicrobial activity by combining, inside liposomes, antimicrobial peptides and fatty acids. In particular, antimicrobial peptides may be selected from the classes of defensins, thionins, heveins, snakins/GASA, knottins. Fatty acids may contain 4 to 22 carbon atoms and may be saturated, monounsaturated or polyunsaturated. The present peptides and fatty acids synergize, thereby providing a strong antimicrobial, in particular antifungal and antibacterial, activity, with important applications, especially in the agronomic field. Selected combinations of fatty acids with antimicrobial peptides are also disclosed herein with additional advantages. Moreover, irrespective of the combination of antimicrobial peptide and fatty acid, the liposome formulation has obtained an unexpected in-vivo resistance to the wash off, resulting in an unexpectedly improved form of application for synergistic combinations of antimicrobial peptides and fatty acids.

### DETAILED DESCRIPTION OF THE INVENTION

A combination of at least one antimicrobial peptide and at least one fatty acid, as active ingredients in a liposome formulation, is object of the present invention. In said liposomes, the above-mentioned ingredients are suitably formulated with excipients and a suitable carrier, in particular for the use in agriculture.

As used herein, the term “liposome” or “liposomes” refers to closed microscopic vesicles having an inner phase enclosed by a lipid bilayer or multilayer. In the present invention, the liposome includes small single-membrane liposomes, large single-membrane liposomes, multilayered liposomes with multiple concentric or non-concentric membranes, etc. The liposome contains an inner phase consisting of an aqueous region enclosed in the lipid bi-/multilayer of the liposome, and an outer phase consisting of the lipid bi-/multilayer. Typically, the lipidic ingredient comprises one or more biocompatible lipids, for example the lipid may be selected from the group consisting of phosphatidylcholine (PC), phosphatidic acid (PA), phosphatidylethanolamine (PE), phosphatidylglycerol (PG), phosphatidylserine (PS), phosphatidylinositol (PI), dimyristoyl-sn-glycero-phosphatidylcholine (DMPC), distearoyl phosphatidylcholine (DSPC), dipalmitoylphosphatidylcholine (DPPC), dimyristoyl phosphatidylglycerol (DMPG), distearoyl phosphatidyl glycerol (DSPG), dioleoyl phosphatidylglycerol (DOPG), dipalmitoylphosphatidylglycerol (DPPG), dimyristoyl phosphatidylserine (DMPS), distearoyl phosphatidylserine (DSPS), dioleoyl phosphatidylserine (DOPS), dipalmitoyl phosphatidylserine (DPPS), dioleoyl phosphatidylethanolamine (DOPE), palmitoyloleoylphosphatidylcholine (POPC), palmitoyl oleoyl phosphatidylethanolamine (POPE), dioleoyl phosphatidylethanolamine 4-(N-maleimidomethyl cyclohexane-1-carboxylate) (DOPE-mal), dipalmitoyl phosphatidyl ethanolamine (DPPE), dimyristoyl phosphatidylethanolamine (DMPE), distearoyl phosphatidyl ethanolamine (DSPE), distearoyl phosphatidylcholine (DSPC), dioleoyl phosphatidylcholine (DOPC), dipalmitoylphosphatidylcholine (DPPC), palmitoyl oleoyl phosphatidylethanolamine (POPE), sphingomyelin, etc.

Moreover, the lipid phase may contain one or more sterols such as, for example, cholesterol, ergosterol, lanosterol, sitosterol, etc.

Mixtures of the above-mentioned non-sterol lipids with sterol lipids are also envisaged, for example mixtures of phosphatidylcholine and/or phosphoglycerol with cholesterol. In an embodiment, the lipidic ingredient may comprise one or more of phosphatidylcholine (PC), phosphatidylinositol (PI), phosphatidylethanolamine (PE), phosphatidic acid (PA), dipalmitoylphosphatidylcholine (DPPC), distearoyl phosphatidylcholine (DSPC), distearoyl phosphatidyl glycerol (DSPG) and cholesterol. In another embodiment, the lipidic ingredient comprises dipalmitoylphosphatidylcholine (DPPC) and cholesterol, or cholesterol and distearoyl phosphatidylcholine (DSPC). In an embodiment, the lipid and the sterol are contained in a molar ratio from about 0.5:1 to about 6:1, preferably about 3:1.

Further lipids and combinations thereof may be freely chosen by the person skilled in the art from those currently used for the preparation of liposomes depending on the type of formulation desired and/or on the availability of materials.

The present liposomes generally have a spherical or similar shape. However, they are not particularly limited in terms of shape as long as they are capable of encapsulating the present combination of peptides and fatty acids, which is herein simply defined as “active ingredient”. The term “encapsulating” means taking a form wherein the active ingredient is contained in an aqueous phase associated to a lipidic membrane: for example, the liposome may be a form wherein the active ingredient is encapsulated inside a closed space formed by the membrane, a form wherein the active ingredient is included in the membrane itself or a combination thereof.

In the scope of the present invention, the term “active ingredient/lipids ratio” refers to the relative amounts by weight of the active ingredient with respect to the lipidic ingredients. In an embodiment, the liposome has an active ingredient:lipid ratio between about 1:1 and about 1:100 by weight. In another embodiment, the liposome has an active ingredient:lipid ratio between about 1:2 and about 1:20 by weight.

The pH can affect the properties of the liposomal formulation, in particular the stability, the rate of outflow of the active ingredient from the liposome and the capability of encapsulation of the active ingredient into the liposome formulation. The pH value of the liposomal formulation is between about 4.0 and about 7.0. In an embodiment, the liposomal formulation has a pH value in the range from about 5.0 to about 6.0.

According to the invention, the liposomal formulation comprises a plurality of liposomes that have the above-described characteristics and are substantially uniform in terms of size and shape. The liposomes may be in the range of size from about 100 to about 10000 nm. In an embodiment, the range of size is from about 1000 to about 5000 nm, in particular, the range of size is from about 1000 to about 3000 nm.

According to the present invention, the liposomal formulation may comprise one or more physiologically acceptable carriers and excipients and auxiliary substances known in the art. The liposome formulation may be administered by any route that effectively transports the liposomes to the suitable site of action, for example, in the agronomic field, by foliar application, fertigation and endotherapy.

According to the invention, the liposomal formulation may comprise one or more additional components selected from: surfactants, dispersing agents, tackifiers, thickeners, antifreezing agents, antimicrobial agents, antioxidants, etc.

Preferably, said additional components are contained in an amount from 0.1% to 10% by weight with respect to the total weight of the composition. Preferably, said surfactants are selected from the anionic or non-ionic surfactants; said anionic surfactant is preferably selected from dodecylbenzenesulfonate and sodiumlaurylsarcosinate; said non-ionic surfactant is preferably selected from ethoxylated tristyrylphenol, ethoxylated fatty alcohol, decyl octyl glycoside. Preferably, said dispersing agent is sodium lignosulfonate. Preferably, said thickener is xanthan gum. Preferably, said antifreezing agent is propylene glycol. Preferably, said antioxidant agent is butylhydroxytoluene (BHT) or another antioxidant selected from water-soluble antioxidants and oil-soluble antioxidants; examples of oil-soluble antioxidants include, but are not limited to, alpha tocopherol, alpha-tocopherol succinate, alpha-tocopherol acetate and mixtures thereof; examples of water-soluble antioxidants include, but are not limited to, ascorbic acid, sodium bisulfite, sodium sulfite, sodium pyrosulfite, L-cysteine and mixtures thereof. The ratio of antioxidant added is from about 0 to about 1.0% (w/v). In an embodiment, the antioxidant is omitted.

The processes for producing the liposomes and the liposome formulation allow to modify the physical characteristics described above, as well as to control some process parameters, for example solvent composition, solvent ratios and the temperature for liposome preparation. The preparation of the liposomal formulation generally comprises the following steps: (1) mixing the active ingredient with lipidic ingredients; (2) heating the mixture to a temperature higher than the transition temperature of the lipid; (3) injecting the mixture in an aqueous medium, for example a saline, to form liposomal vesicles.

More specifically, the first step comprises solubilizing the lipid or mixtures thereof with a solvent to form a lipidic solution. The solution is heated to a temperature suitable for facilitating the solubilization of the lipid or mixtures thereof, for example in the range from about 40° C. to about 80° C., for example 60° C. The second step consists in injecting the mixture in an aqueous medium, for example water or a saline containing the polypeptide and/or active ingredient for forming the liposomes at room temperature, for example at a temperature of about 25° C., or possibly at a higher temperature. If desired, a step of ultrafiltration and concentration of the resulting solution containing the liposome may be carried out, using suitable types of filtration membranes.

Other preparation methods are possible and are described in the literature of the art, for example in Comprehensive Reviews in Food Science and Food Safety, 2021, 20, 1280-1306, or Nanoscale Res Lett. 2013; 8(1): 102. Published online 2013 Feb. 22. doi: 10.1186/1556-276X-8-102, herein incorporated by reference.

The present combinations of antimicrobial peptides and fatty acids have unexpectedly benefited from their liposome formulation in that they are made strongly more resistant to water leaching after their application to the plant. As documented by the data in the experimental section, at constancy of other ingredients, the liposome formulation obtains a 2-6-fold reduction of the leaching process, depending on the particular antimicrobial peptide used.

Any peptide with antimicrobial activity is suitable for being included in the present liposomes. Antimicrobial peptides are per se widely known and described in the literature. In the present invention, peptides belonging to the classes of defensins, thionins, heveins, snakins/GASA, knottins proved to be very effective.

Defensins are a phylogenetically very old peptide family, with a highly conserved structure, that are present in mammals, insects and plants: they are amphipathic peptides capable of inserting in membranes and of inducing the pore formation resulting in death due to cells lysis. There are two main defensin categories: α and β and they differ in the type of producing cell and thus in localization. α defensins are mainly produced by neutrophils (contained in primary granules) and by Paneth cells; they are produced and secreted as an inactive form of pro-peptide and are activated by proteolytic cleavage by trypsin. β defensins are produced by epithelial cells of the respiratory system, the integumentary system, the urogenital system and the skin.

An interesting subclass of antimicrobial peptides is the one of plant defensins (Planta, 2002, 216, pp-193-202). Particularly interesting peptides among them are the following:
Hs-AFP1, corresponding to SEQ.ID.NO: 1
Rs-AFP2, corresponding to SEQ.ID.NO: 2
Ah-AMP1, corresponding to SEQ.ID.NO: 3
NmDef2, corresponding to SEQ.ID.NO: 4
Oh-DEF, corresponding to SEQ.ID.NO: 5
DefMT6, corresponding to SEQ.ID.NO: 6
AvBD1, corresponding to SEQ.ID.NO: 7
mDB14, corresponding to SEQ.ID.NO: 8
PsDefl, corresponding to SEQ.ID.NO: 9
Def-Tk, corresponding to SEQ.ID.NO: 10
Abf-2, corresponding to SEQ.ID.NO: 11
K7MPK0, corresponding to SEQ.ID.NO: 12
Def1.1, corresponding to SEQ.ID.NO: 13
OsDef8 corresponding to SEQ.ID.NO: 14
Termicin, corresponding to SEQ.ID.NO: 15

The peptides referred herein are per se known; for example, the peptide Hs-AFP1 is per se known from WO200472239, WO202186982 and WO2016205902; the peptide Rs-AFP2 is per se known e.g. from WO200109174 and WO200109175.

Another class of antimicrobial peptides that is particularly effective in the present invention is the class of heveins. They are peptides originating from the rubber tree (Hevea brasiliensis), that are obtained from the incision of the tree and have properties promoting latex coagulation. Heveins are the result of hydrolysis of the natural peptide (pro-hevein, containing 187 amino acids) into shorter fragments. Preferred examples of heveins that can be used in the present invention are the peptides:
Ay-AMP, corresponding to SEQ.ID.NO: 16;
Ee-CBP, corresponding to SEQ.ID.NO: 17.

A further class of antimicrobial peptides that is particularly effective in the present invention is the class of snakins (also identified as GASA family). Snakins are plant antimicrobial peptides consisting of three distinct regions: an N-terminal signal peptide; a variable site; and the GASA domain in the C-terminal region composed of twelve cysteine residues that contribute to the biochemical stability of the molecule. These peptides are known to play different roles in response to a variety of stress factors. A preferred example of snakins that can be used in the present invention is the peptide:
StSN1, corresponding to SEQ.ID.NO: 18.

A further class of antimicrobial peptides that is particularly effective in the present invention is the class of knottins (cystine-knots (ICKs)). They are peptides characterized in that they contain three disulfide bridges, that form an intramolecular knot and give structural and functional resistance to high temperatures, to enzymatic degradation, to extreme pH and to mechanical stresses. The loops connecting the disulfide bridges show a high sequence variability, resulting in a wide range of functions. A preferred example of knottins that can be used in the present invention is the peptide:
McAMP1, corresponding to SEQ.ID.NO: 19.

Another class of antimicrobial peptides that is particularly effective in the present invention is the class of thionins. An important subclass thereof is the subclass of viscotoxins (Biophysical Journal Volume 85 Aug. 2003 971-981). Among them, a peptide useful for the purposes of the invention is:
VtA3, corresponding to SEQ.ID.NO: 20.

A subgroup of peptide preferred according to the invention is the subgroup consisting of Hs-AFP1, Rs-AFP2 or PsDef-1, which have the above-mentioned structures.

The fatty acids that can be used in the present composition may be indifferently saturated, monounsaturated or polyunsaturated, being preferably selected in the interval C₄-C₂₂. Said fatty acids can be used as such and/or in the form of salts thereof and/or in the form of hydroxylated derivatives thereof; said variants are all included in the definition of “fatty acids” according to the present invention. Specific preferred fatty acids are butyric acid, pelargonic acid, crotonic acid, caproleic acid, oleic acid, linoleic acid; particularly preferred are: butyric acid, pelargonic acid, oleic acid, linoleic acid. The fatty acids used in the invention can either have or not have antimicrobial activity per se: in any case they synergically interact with the peptide, thereby causing an overall antimicrobial activity higher than the sum of the activities of the two components taken separately.

A preferred sub-embodiment of the present invention is represented by new combinations of defensins with fatty acids selected from the group consisting of crotonic acid, pelargonic acid, caproleic acid and mixtures thereof. These combinations have shown a remarkably high level of synergic antimicrobial interaction (measured as FIC Index) against a large variety of target microorganisms, including fungi, Gram positive and Gram negative bacteria, inclusive of phytoplasma, thus conjugating the hardly conciliable effects of non-specificity and efficacy; the highest level of synergy is present when the defensins are combined with pelargonic acid, which represents an even more preferred combination. The obtained high levels of synergy are paralleled by a high level of antiinfective activity when applied on plant infections in open field, as confirmed by the experimental data included in this specification. A further remarkable advantage of the combinations according to this sub-embodiment is their potent inhibitory activity against phytoplasms, a sub-class of bacteria responsible for hardly curable plant diseases, being resistant to conventional antibacterial agents; the level of activity is dramatically higher compared to that shown by the two separate products tested alone: this indicates that the synergism among these agents is especially amplified when the target microorganism is a phytoplasma; this is particularly unexpected because antimicrobial peptides and fatty acids are known to exert antimicrobial activity via interaction with the bacterial cell wall, a cellular component absent in the phytoplasma. In the present sub-embodiment, any defensin can be used in combination with said crotonic, pelargonic and/or caproleic acid; examples of suitable defensins are: Hs-AFP1, corresponding to SEQ.ID.NO: 1; Rs-AFP2, corresponding to SEQ.ID.NO: 2; Ah-AMP1, corresponding to SEQ.ID.NO: 3; NmDef2, corresponding to SEQ.ID.NO: 4; Oh-DEF, corresponding to SEQ.ID.NO: 5; DefMT6, corresponding to SEQ.ID.NO: 6; AvBD1, corresponding to SEQ.ID.NO: 7; mDB14, corresponding to SEQ.ID.NO: 8; PsDefl, corresponding to SEQ.ID.NO: 9; Def-Tk, corresponding to SEQ.ID.NO: 10; Abf-2, corresponding to SEQ.ID.NO: 11; K7MPK0, corresponding to SEQ.ID.NO: 12; Def1.1, corresponding to SEQ.ID.NO: 13; OsDef8 corresponding to SEQ.ID.NO: 14; Termicin, corresponding to SEQ.ID.NO: 15. Particularly preferred are the combinations of crotonic acid, pelargonic acid and/or caproleic acid with one or more of said Hs-AFP1, Rs-AFP2 and PsDefl.

In all embodiments of the present invention, the peptides and the fatty acids can be combined with each other in all the possible proportions; preferably, neither of the two components is used in a weight ratio with respect to the other lower than 1:9. More preferably, the peptide (or their mixture, if more than one of them are used) is contained in a weight ratio with the fatty acid (or their mixture, if more than one of them are used) between 0.3:1 and 0.5:1; or alternatively between 0.5:1 and 1.5:1, for example in a 1:1 ratio.

In the present invention, the association of antimicrobial peptides with fatty acids obtains very high synergy levels, i.e. characterized by FIC index 0.7, preferably between 0.05 and 0.5. According to the standard literature, the FIC Index can be calculated with the following formula:

$$FIC index = MICA / MICa + MICB / MICb$$

Wherein “MICA and MICB” are the minimum inhibitory concentrations (MIC) of the two compounds A and B mixed with each other whereas “MICa and MICb” are the minimum inhibitory concentrations of the two components used singularly. FIC index<1.0 means synergy of the compounds combined with each other; FIC index=1.0 means that the compounds do not interact with each other; FIC index>1.0 means antagonism of the compounds combined with each other.

The term “antimicrobial” used herein is to be understood as comprising the terms antifungal, antibacterial, antiviral and antiparasitic. Preferably, the antimicrobial treatment is an antifungal or antibacterial treatment.

For the purposes of the antifungal treatment, all the fungal species can be treated according to the invention. Among them, the species preferably recommended for the purposes of the present treatment are the following.

In the agronomic field: Botrytis cinerea, Fusarium culmorum, Fusarium graminearum, Fusarium oxysporum, Fusarium solani, Stemphylium vesicarium, Scleratium rolfsii, Bipolaris sorokiniana, Sclerotinia sclerotiorum, Rhizoctonia solani, Zymoseptoria tritici, Cercospora beticola, Alternaria alternata, Venturia inequalis, Magnaporthe oryzae, Phytophtora infestans, Plasmopara viticola, Phakopsora pachyrhizi, Plasmopara viticola, Taphrina deformans, Uncinula necator, Erysiphe spp. Particularly preferred for the purposes of said treatment are the species: Botrytis cinerea, Fusarium culmorum, Fusarium graminearum, Phytophtora infestans, Alternaria alternata, Venturia inequalis.

In the pharmaceutical, nutraceutical or cosmetic field: Candida albicans, Aspergillus fumigatus, Cryptococcus neoformans, Malassezia furfur, Trichosporon spp, Histoplasma capsulatum, Blastomyces dermatitidis, Coccidioides immitis.

For the purposes of the antibacterial treatment, all the bacterial species, including phytoplasmas, can be treated according to the invention. Among them, the species preferably recommended for the purposes of the present treatment are the following.

In the agronomic field: Erwinia amylovora, Pseudomonas syringae, Xanthomonas campestris, Xanthomonas phaseoli, Xylella fastidiosa. Phytoplasmas that may be mentioned are for example: ‘Ca. Phytoplasma castaneae’, ‘Ca. Phytoplasma graminis’, ‘Ca. Phytoplasma japonicum’, ‘Ca. Phytoplasma lycopersici’, ‘Ca. Phytoplasma oryzae’, ‘Ca. Phytoplasma pruni’, ‘Ca. Phytoplasma pyri’, ‘Ca. Phytoplasma solani’, ‘Ca. Phytoplasma vitis’.

In the pharmaceutical, nutraceutical or cosmetic field: Pseudomonas aeruginosa, Staphylococcus aureus, Escherichia coli, Campylobacter jejuni, Bacillus cereus, Listeria monocytogenes, Salmonella typhimurium, Clostridium perfringens. Preferably, bacterial species that can be treated are: Escherichia coli, Staphylococcus aureus, Pseudomonas aeruginosa.

In the present invention, the above-mentioned peptides and fatty acids are formulated in the form of liposomes in conjunction with a carrier suitable for dispersing the resulting composition on a substrate that needs it, for example an aerial part of a plant that can be treated by superficial or endotherapic application or an agricultural land. Said composition, including the carrier, may be solid, semisolid or, preferably, liquid. Solid compositions may, for example, be in the form of powders, pellets, granules, microcapsules, etc.: said solid compositions may be delivered as such or may be previously dispersed in a liquid medium before administration on the land or on the plant. Semisolid compositions may be in the form of creams, pastes, gels, hydrogels, and the like. Liquid compositions may be in the form of a solution, suspension, dispersion, colloid, emulsion, etc.; they may be administered as such or in the form of an aerosol or spray. Depending on their physical form, on the nature of the active ingredients contained, and on the usage conditions, the present compositions may include, in addition to the above-mentioned peptides, fatty acids and carrier, further excipients and other co-formulation agents according to the prior art of the field; in particular, they can contain stabilizers, antioxidants, buffering agents, chelating agents, agents for controlling the pH for example buffer systems, isotonicity agents, emulsifiers, co-emulsifiers, thickeners, gelling agents, film-forming agents, lubricants, glidants, anti-aggregating agents, moisture absorbers, coloring agents, etc.

Depending on their physical form and on the treatment needs (plant type and/or land type), the present compositions may be administered as such or dispersed in water, in fertilizing solutions, in biostimulating solutions, etc. For the purposes of an effective treatment, it is useful that the composition is administered in such an amount to provide a dose of mixture [peptide+fatty acid]/hectare (ha) of land between 50 Kg and 0.1 Kg, preferably between 5 Kg and 1 Kg.

A further object of the present invention is the use of a liposomal composition as defined above, comprising one or more antimicrobial peptides and one or more fatty acids, as an antimicrobial, preferably antifungal, agent. The present compositions can be used for both a preventive and a curative purpose, depending on the needs. The use is preferably intended in the agronomic field; however, the present association of peptides and fatty acids is also active in different fields and can be used without limitation for any antimicrobial treatment: said applications are equally part of the present invention. Therefore, the invention comprises also the preparation, provision and use of the present compositions in the pharmaceutical, nutraceutical or cosmetic field; the excipients and co-formulation agents used in these variants will be the one suitable for the respective pharmaceutical, nutraceutical, cosmetic use. A further object of the present invention is the use of one or more antimicrobial peptides and one or more fatty acids as defined above, in the preparation of an antimicrobial, preferably antifungal, composition in the form of liposomes.

A further object of the present invention is a process for the preparation of an antimicrobial composition with high synergistic activity, in the form of liposomes, preferably for agronomic use, said process comprising formulating with each other: one or more antimicrobial peptides as defined above, one or more fatty acids as defined above, and, optionally, a suitable carrier and/or co-formulation agents.

The present invention is now described by way of the following non-limiting examples.

### Experimental Part

### Example 1—Evaluation of Synergism

### General Procedure

The antimicrobial activity was evaluated using the in-vitro susceptibility test with the microdilution method described in the Clinical and Laboratory Standard Institute protocols (M07—Methods for Dilution Antimicrobial Susceptibility Test for Bacteria That Grow Aerobically; M27—Reference Method for Broth Dilution Antifungal Susceptibility Testing of Yeasts; Reference Method for Broth Dilution Antifungal Susceptibility Testing of Filamentous Fungi). The MIC (Minimum Inhibitory Concentration) for each compound of interest was determined by these methods.

The positive control of the antimicrobial activity was carried out using fluconazole (for fungi and yeasts) and ceftriaxone (for bacterial strains). The negative control (absence of the active compounds) was evaluated by observing the correct microbial growth of the species of interest.

The synergistic activity of the various compounds was evaluated in vitro with the microdilution method in 96-well plates. Samples of the compounds of interest were prepared by diluting said compounds in sterile physiological solution to a specific concentration of 4 times the previously determined MIC. Then, combinations at different concentrations of the antimicrobial peptides with the fatty acids were made and said samples were treated as described in the CLSI protocols.

The synergy between the peptides and the fatty acids according to the invention was evaluated by calculating the FIC index according to the following formula:

$$FIC index = MICA / MICa + MICB / MICb$$

wherein “MICA and MICB” are the minimum inhibitory concentrations (MIC) of the two compounds A and B mixed with each other whereas “MICa and MICb” are the minimum inhibitory concentrations of the two components used singularly.

A value of FIC index<1.0 means synergy of the compounds combined with each other; a value of FIC index=1.0 means the absence of synergy of the compounds combined with each other; a value of FIC index>1.0 means antagonism of the compounds combined with each other.

The experimental results obtained are shown in Table I and in

TABLE II
Table I—Antifungal activity

| Product A | Product B | FIC | |
|---|---|---|---|
| (Peptide) | (Fatty acid) | INDEX | Microorganism |
| Hs-AFP1, | Pelargonic acid | 0.1-0.3 | Botrytis cinerea |
| Rs-AFP2 or | Crotonic acid | 0.5-0.7 | Fusarium culmorum |
| PsDef1 | Caproleic acid | 0.5-0.7 | Fusarium |
| | | | graminearum |
| | | | Bipolaris sorokiniana |
| | | | Sclerotinia |
| | | | sclerotiorum |
| | | | Rhizoctonia solani |
| | | | Zymoseptoria tritici |
| | | | Cercospora beticola |
| | | | Alternaria alternata |
| | | | Venturia inequalis |
| | | | Magnaporthe oryzae |
| | | | Phytophtora infestans |
| | | | Plasmopara viticola* |
| | | | Phakopsora |
| | | | pachyrhizi* |

TABLE II
Antibacterial activity

| | Product B | FIC | |
|---|---|---|---|
| Product A (Peptide) | (Fatty acid) | INDEX | Microorganism |
| Hs-AFP1, Rs-AFP2 | Pelargonic acid | 0.1-0.3 | E. coli |
| or PsDef1 | Crotonic acid | 0.4-0.7 | S. aureus |
| | Caproleic acid | 0.4-0.7 | P. aeruginosa |

### Example 2

Open Field Testing—Antifungal Activity on Fusarium graminearum by Mixtures of Fatty Acids and Peptides

The antifungal activity of three peptides (SEQ.ID.NOs.: 1, 2 and 9), of the crotonic and pelargonic acid and of the mixtures of these fatty acids with the aforementioned peptides was evaluated on winter wheat and durum wheat suitably contaminated by Fusarium graminearum.

The peptides of SEQ.ID.NOs.: 1, 2 and 9 were dissolved in water at a concentration of 10% w/w. Aqueous solutions of crotonic and pelargonic acids were prepared at a concentration of 10% w/w. Aqueous solutions of peptides and solutions of fatty acids were mixed together, in order to obtain six different mixtures at a concentration of 10% w/w of peptide and acid. The solutions were used at the dosages indicated in the table on both cultivars, after 2 days from the inoculation of the pathogenic strain Fusarium graminearum. Only one application was carried out at the time corresponding to the phenological scale BBCH (Biologische Bundesanstalt, Bundessortenamt and CHemical industry) 69-70 and the efficacy of the products was evaluated after 7 days for 3 weeks starting from the last check. The efficacy was assessed as incidence of leaves affected by the target pathogen compared to the untreated control: an increased % efficacy corresponds to a decreased number of leaves infested with the nhytopathogen.

| | | Efficacy (%) | | |
|---|---|---|---|---|
| Products | Concentration | 1° check | 2° check | 3° check |
| Hs_AFP1 | 2 L/ha | 35 | 27.4 | 26.5 |
| Rs_AFP2 | 2 L/ha | 38 | 34 | 25.4 |
| Ps_Def1 | 2 L/ha | 30 | 27 | 32 |
| Pelargonic acid | 2 L/ha | 10 | 8 | 9 |
| Crotonic acid | 2 L/ha | 8 | 7 | 10 |
| Hs_AFP1 + | 1 L/ha + | 84 | 82 | 78 |
| Pelargonic acid | 1 L/ha | | | |
| Rs_AFP2 + | 1 L/ha + | 78 | 75 | 72 |
| Pelargonic acid | 1 L/ha | | | |
| Ps_Def1 + | 1 L/ha + | 78 | 72 | 73 |
| Pelargonic acid | 1 L/ha | | | |
| Hs_AFP1 + | 1 L/ha + | 80 | 77 | 75 |
| Crotonic acid | 1 L/ha | | | |
| Rs_AFP2 + | 1 L/ha + | 76 | 73 | 69 |
| Crotonic acid | 1 L/ha | | | |
| Ps_Def1 + | 1 L/ha + | 73 | 67 | 64 |
| Crotonic acid | 1 L/ha | | | |
| Reference commercial | 1 L/ha | 88 | 84 | 73 |
| product | | | | |
| Untreated | 0 | 0 | 0 | 0 |

### Example 3

Open Filed Testing on Phytoplasma—Antibacterial Activity on Ca. Phytoplasma vitis by Mixtures of Fatty Acids and Peptides

The activity on phytoplasma of three peptides (SEQ.ID.NO.: 1, 2 and 9), of crotonic and pelargonic acid and of the mixtures of these fatty acids with the aforementioned peptides were evaluated on Pervinca rosea suitably contaminated by Ca. Phytoplasma vitis.

The peptides of SEQ.ID.NO.: 1, 2 and 9 were dissolved in water at a concentration of 10% w/w. Aqueous solutions of crotonic and pelargonic acids were prepared at a concentration of 10% w/w. Aqueous solutions of peptides and solutions of fatty acids were mixed together, in order to obtain six different mixtures at a concentration of 10% w/w of peptide and acid.

The evaluation of the efficacy of the products used was evaluated with ddPCR (Droplet Digital PCR) technique for the quantification of the genetic material (DNA and RNA) after 48 hours from exposure to endotherapy treatment. High % DNA and RNA values are related to reduced/no antibacterial activity. The solutions were used through endotherapic treatment at the dosages indicated in the following table:

| | | Efficacy(%) | |
|---|---|---|---|
| Sample | Concentration | % DNA | % RNA |
| Hs_AFP1 | 2 L/ha | 75 | 78 |
| Rs_AFP2 | 2 L/ha | 81 | 80 |
| Ps_Def1 | 2 L/ha | 82 | 80 |
| Crotonic acid | 2 L/ha | 76 | 84 |
| Pelargonic acid | 2 L/ha | 84 | 86 |
| Hs_AFP1 + crotonic acid | 1 L/ha + 1 L/ha | 42 | 48 |
| Rs_AFP2 + crotonic acid | 1 L/ha + 1 L/ha | 46 | 54 |
| Ps_Def1 + crotonic acid | 1 L/ha + 1 L/ha | 44 | 52 |
| Hs_AFP1 + pelargonic acid | 1 L/ha + 1 L/ha | 45 | 50 |
| Rs_AFP2 + pelargonic acid | 1 L/ha + 1 L/ha | 50 | 60 |
| Ps_Def1 + pelargonic acid | 1 L/ha + 1 L/ha | 52 | 52 |
| Untreated | 0 | 91 | 94 |

### Example 4—Liposomal Preparation A

10 g sunflower lecithin (SternPur SP200 Sternchemie) were mixed with 15 g propylene glycol and stirred for 10 minutes at 200 rpm. The mixture thereby obtained is supplemented, at room temperature, with an aqueous solution of 1.0 g polypeptide (Seq. 1) and 0.1 g butyric acid and stirred for 30 minutes at 200 rpm.

The liposomes thereby obtained are separated by centrifugation.

### Example 5—Liposomal Preparation B

10 g sunflower lecithin (SternPur SP200 Sternchemie) were mixed with 15 g propylene glycol and 2.5 g cholesterol and stirred for 10 minutes at 200 rpm. The mixture thereby obtained is supplemented, at room temperature, with an aqueous solution of 1.0 g polypeptide (Seq.9) and 0.1 g crotonic acid and stirred for 30 minutes at 200 rpm.

The liposomes thereby obtained are separated by centrifugation.

### Example 6—Liposomal Preparation C-L

Similarly to what is described in example 4, with the same quantities of reagents, except replacing butyric acid with crotonic acid where indicated, the following liposomes were prepared starting from peptides of the sequences indicated.

| Liposome | Used peptide | Used acid |
|---|---|---|
| preparation | (g) | (g) |
| 4C | Seq. 17 | Butyric |
| | (1.0) | (0.1) |
| 4D | Seq. 18 | Butyric |
| | (1.0) | (0.1) |
| 4E | Seq. 19 | Butyric |
| | (1.0) | (0.1) |
| 4F | Seq. 20 | Butyric |
| | (1.0) | (0.1) |
| 4G | Seq. 17 | Crotonic |
| | (1.0) | (0.1) |
| 4H | Seq. 18 | Crotonic |
| | (1.0) | (0.1) |
| 4I | Seq. 19 | Crotonic |
| | (1.0) | (0.1) |
| 4L | Seq. 20 | Crotonic |
| | (1.0) | (0.1) |

The liposomes of the previous examples were tested to verify their residuality after washing with water.

### Example 7—Residuality of the Peptides and Fatty Acids Contained in the Liposomes of Example 6 (C-L) (In Vitro Test)

The liposomes of example 6 (C-L) are tested in vitro to determine their resistance to leaching.

A section of Parafilm® thermoplastic film (10×30 cm) is treated by spraying with 2 mL of a solution obtained by diluting the liposome in water at 40% w/v.

The thermoplastic film section is allowed to dry at room temperature and the entire surface is then washed with 12 mL of water using a standard spray system with 1 mm nozzle. After washing, the thermoplastic film section is dried and then treated with an 8:2 water/acetone solution. The collected solution is analysed to determine the residual quantity of the products after washing by HPLC-MS technique.

The results are reported in Tables IIIA and IIIB.

TABLE IIIA

| | | | Residual | | Residual |
|---|---|---|---|---|---|
| | | | peptide | | crotonic |
| | | | amount | Crotonic | acid |
| | | Peptide | (% of | acid | amount |
| | | amount | initial | amount | (% of |
| | | (mg) | value) | (mg) | initial value) |
| Liposome | seq. 1 + | 32 | 65 | 3.2 | 73 |
| formulation | crotonic acid | | | | |
| | seq. 17 + | 32 | 58 | 3.2 | 75 |
| | crotonic acid | | | | |
| | seq. 18 + | 32 | 63 | 3.2 | 68 |
| | crotonic acid | | | | |
| | seq. 19 + | 32 | 59 | 3.2 | 73 |
| | crotonic acid | | | | |
| | seq. 20 + | 32 | 61 | 3.2 | 69 |
| | crotonic acid | | | | |
| Liquid | seq. 1 + | 32 | 27 | 3.2 | 40 |
| formulation | crotonic acid | | | | |
| | seq. 17 + | 32 | 23 | 3.2 | 42 |
| | crotonic acid | | | | |
| | seq. 18 + | 32 | 20 | 3.2 | 37 |
| | crotonic acid | | | | |
| | seq. 19 + | 32 | 13 | 3.2 | 31 |
| | crotonic acid | | | | |
| | seq. 20 + | 32 | 14 | 3.2 | 32 |
| | crotonic acid | | | | |

TABLE IIIB

| | | | Residual | | Residual |
|---|---|---|---|---|---|
| | | | peptide | | butyric |
| | | | amount | Butyric | acid |
| | | Peptide | (% of | acid | amount |
| | | amount | initial | amount | (% of |
| | | (mg) | value) | (mg) | initial value) |
| Liposome | seq. 1 + | 32 | 62 | 3.2 | 75 |
| formulation | butyric acid | | | | |
| | seq. 17 + | 32 | 65 | 3.2 | 74 |
| | butyric acid | | | | |
| | seq. 18 + | 32 | 56 | 3.2 | 70 |
| | butyric acid | | | | |
| | seq. 19 + | 32 | 54 | 3.2 | 72 |
| | butyric acid | | | | |
| | seq. 20 + | 32 | 61 | 3.2 | 70 |
| | butyric acid | | | | |
| Liquid | seq. 1 + | 32 | 25 | 3.2 | 38 |
| formulation | butyric acid | | | | |
| | seq. 17 + | 32 | 20 | 3.2 | 40 |
| | butyric acid | | | | |
| | seq. 18 + | 32 | 18 | 3.2 | 36 |
| | butyric acid | | | | |
| | seq. 19 + | 32 | 12 | 3.2 | 32 |
| | butyric acid | | | | |
| | seq. 20 + | 32 | 15 | 3.2 | 30 |
| | butyric acid | | | | |

### Example 8—Residuality of the Peptides Contained in the Liposomes of Example 6 (C-L) after Treatment on the Plant

Plots of 5 m² are set up containing BBCH 13-29 tomato seedlings grown in greenhouses in order to evaluate the residuality of the peptides (most water-soluble component) formulated in liposomes obtained according to example 6 (C-L). As a control, the same products not formulated into liposomes are used at the same dose.

The trial design is shown in the following tables IVA and JVB.

TABLE IVA

| | Number of | Number of | |
|---|---|---|---|
| Thesis | applications | replicates | Dose |
| Liposome formulation | | | |
| seq. 1 + crotonic acid | 1 | 3 | 4 L/ha |
| seq. 17 + crotonic acid | 1 | 3 | 4 L/ha |
| seq. 18 + crotonic acid | 1 | 3 | 4 L/ha |
| seq. 19 + crotonic acid | 1 | 3 | 4 L/ha |
| seq. 20 + crotonic acid | 1 | 3 | 4 L/ha |
| Liquid formulation | | | |
| seq. 1 + crotonic acid | 1 | 3 | 4 L/ha |
| seq. 17 + crotonic acid | 1 | 3 | 4 L/ha |
| seq. 18 + crotonic acid | 1 | 3 | 4 L/ha |
| seq. 19 + crotonic acid | 1 | 3 | 4 L/ha |
| seq. 20 + crotonic acid | 1 | 3 | 4 L/ha |

TABLE IVB

| | Number of | Number of | |
|---|---|---|---|
| Thesis | applications | replicates | Dose |
| Liposome formulation | | | |
| seq. 1 + butyric acid | 1 | 3 | 4 L/ha |
| seq. 17 + butyric acid | 1 | 3 | 4 L/ha |
| seq. 18 + butyric acid | 1 | 3 | 4 L/ha |
| seq. 19 + butyric acid | 1 | 3 | 4 L/ha |
| seq. 20 + butyric acid | 1 | 3 | 4 L/ha |
| Liquid formulation | | | |
| seq. 1 + butyric acid | 1 | 3 | 4 L/ha |
| seq. 17 + butyric acid | 1 | 3 | 4 L/ha |
| seq. 18 + butyric acid | 1 | 3 | 4 L/ha |
| seq. 19 + butyric acid | 1 | 3 | 4 L/ha |
| seq. 20 + butyric acid | 1 | 3 | 4 L/ha |

The liposomal formulations containing the peptides at 4% concentration and the non-liposomal formulations containing the same amount of peptides were applied at a dosage of 4 L/ha by diluting the product in sufficient water to wet the culture well but limiting dripping to a minimum (minimum 200 L/ha).

After 4 hours the seedlings are watered by sprinkling for 5 minutes, to simulate a rain of 40 to 50 mm, then allowing the leaves to dry. The leaves of each thesis, after drying, are collected and analyzed to quantify at the foliar level the peptides related to the liposome-based formulations and soluble liquid formulations.

The results are reported in the tables IVC and IVD.

TABLE IVC

| | | Peptide | Residual peptide |
|---|---|---|---|
| | | amount | amount |
| | | (mg/m²) | (% of initial value) |
| Liposome | seq. 1 + crotonic acid | 16 | 58 |
| formulation | seq. 17 + crotonic acid | 16 | 62 |
| | seq. 18 + crotonic acid | 16 | 51 |
| | seq. 19 + crotonic acid | 16 | 52 |
| | seq. 20 + crotonic acid | 16 | 58 |
| Liquid | seq. 1 + crotonic acid | 16 | 25 |
| formulation | seq. 17 + crotonic acid | 16 | 22 |
| | seq. 18 + crotonic acid | 16 | 23 |
| | seq. 19 + crotonic acid | 16 | 12 |
| | seq. 20 + crotonic acid | 16 | 10 |

TABLE IVD

| | | Peptide | Residual peptide |
|---|---|---|---|
| | | amount | amount |
| | | (mg/m²) | (% of initial value) |
| Liposome | seq. 1 + butyric acid | 16 | 58 |
| formulation | seq. 17 + butyric acid | 16 | 64 |
| | seq. 18 + butyric acid | 16 | 53 |
| | seq. 19 + butyric acid | 16 | 50 |
| | seq. 20 + butyric acid | 16 | 55 |
| Liquid | seq. 1 + butyric acid | 16 | 25 |
| formulation | seq. 17 + butyric acid | 16 | 21 |
| | seq. 18 + butyric acid | 16 | 23 |
| | seq. 19 + butyric acid | 16 | 11 |
| | seq. 20 + butyric acid | 16 | 13 |

The present data show, on one side, that the modification of the liquid formulation into liposomes did not cause any loss of adhesion of the active principle to the plant surface in the application phase. After the watering cycle, the liposome formulation resulted strongly more protected against water leaching. The reduced leaching guarantees a prolonged contact of the active agents with the plant surface, posing the premises for a more efficient and effective treatment of plant infections.

1.-17. (canceled)

18. Liposomes comprising an antimicrobial peptide selected from the class of defensins and a fatty acid selected from the group consisting of crotonic acid, pelargonic acid, caproleic acid and mixtures thereof.

19. The liposomes of claim 18, wherein said defensins are selected from the group consisting of: Hs-AFP1, corresponding to SEQ.ID.NO: 1; Rs-AFP2, corresponding to SEQ.ID.NO: 2; Ah-AMP1, corresponding to SEQ.ID.NO: 3; NmDef2, corresponding to SEQ.ID.NO: 4; Oh-DEF, corresponding to SEQ.ID.NO: 5; DefMT6, corresponding to SEQ.ID.NO: 6; AvBD1, corresponding to SEQ.ID.NO: 7; mDB14, corresponding to SEQ.ID.NO: 8; PsDefl, corresponding to SEQ.ID.NO: 9; Def-Tk, corresponding to SEQ.ID.NO: 10; Abf-2, corresponding to SEQ.ID.NO: 11; K7MPK0, corresponding to SEQ.ID.NO: 12; Def1.1, corresponding to SEQ.ID.NO: 13; OsDef8 corresponding to SEQ.ID.NO: 14; and Termicin, corresponding to SEQ.ID.NO: 15.

20. The liposomes of claim 18, wherein the antimicrobial peptide and fatty acid comprise the active ingredient of the liposomes, and the active ingredient: lipid ratio of the liposomes is between about 1:1 and about 1:100 by weight.

21. The liposomes of claim 20, wherein the active ingredient: lipid ratio of the liposomes is between about 1:2 and about 1:20 by weight.

22. The liposomes of claim 18, wherein the liposomes further comprise an additional component selected from the group consisting of surfactants, dispersing agents, tackifiers, thickeners, antifreezing agents, antimicrobial agents, antioxidants, and mixtures thereof.

23. The liposomes of claim 22, wherein the additional component is present in an amount of from 0.1% to 10% by weight with respect to the total weight of the liposomes.

24. A method of antimicrobial treatment, in the therapeutic or agronomic field, comprising administering the liposomes of claim 18 to a human or other mammal or to land or plants in need of such treatment.

25. The method of claim 24, wherein the antimicrobial treatment is for treating or preventing contaminations by fungi and/or bacteria and/or phytoplasma.

26. The method of claim 25, wherein said fungi are selected from the group consisting of Botrytis cinerea, Fusarium culmorum, Fusarium graminearum, Fusarium oxysporum, Fusarium solani, Stemphylium vesicarium, Scleratium rolfsii, Bipolaris sorokiniana, Sclerotinia sclerotiorum, Rhizoctonia solani, Zymoseptoria tritici, Cercospora beticola, Alternaria alternata, Venturia inequalis, Magnaporthe oryzae, Phytophtora infestans, Plasmopara viticola, Phakopsora pachyrhizi, Plasmopara viticola, Taphrina deformans, Uncinula necator, Erysiphe spp., Candida albicans, Aspergillus fumigatus, Cryptococcus neoformans, Malassezia furfur, Trichosporon spp, Histoplasma capsulatum, Blastomyces dermatitidis, and Coccidioides immitis.

27. The method of claim 25, wherein said bacteria are selected from the group consisting of Erwinia amylovora, Pseudomonas syringae, Xanthomonas campestris, Xanthomonas phaseoli, Xylella fastidiosa, Pseudomonas aeruginosa, Staphylococcus aureus, Escherichia coli, Campylobacter jejuni, Bacillus cereus, Listeria monocytogenes, Salmonella typhimurium, Clostridium perfringens, or phytoplasmas ‘Ca. Phytoplasma castaneae’, ‘Ca. Phytoplasma graminis’, ‘Ca. Phytoplasma japonicum’, ‘Ca. Phytoplasma lycopersici’, ‘Ca. Phytoplasma oryzae’, ‘Ca. Phytoplasma pruni’, ‘Ca. Phytoplasma pyri’, ‘Ca. Phytoplasma solani’, and ‘Ca. Phytoplasma vitis’.

28. A process for the preparation of the liposomes of claim 18, comprising formulating with each other: an antimicrobial peptide selected from the class of defensins, and a fatty acid selected from the group consisting of crotonic acid, pelargonic acid, caproleic acid and mixtures thereof, and one or more lipid agents under conditions suitable for forming lipid vesicles encapsulating said peptides and fatty acids.

29. A composition comprising the liposomes of claim 18, in conjunction with one or more carriers and excipients.

30. A process for preparing the composition of claim 29, comprising formulating liposomes comprising an antimicrobial peptide selected from the class of defensins and a fatty acid selected from the group consisting of crotonic acid, pelargonic acid, caproleic acid and mixtures thereof and one or more carriers and excipients with each other.
